(19) United States
(12) Patent Application Publication
(10) Pub. No.: US 2008/0167266 A1
(43) Pub. Date: Jul. 10, 2008

(54) Process For Preparing An Iron Saccharose Complex

(76) Inventor: Michael Justus, Schaffhausen (CH)

Correspondence Address: HAMMER & HANF, PC, 3125 SPRINGBANK LANE, SUITE G, CHARLOTTE, NC, 28226, US

(21) Appl. No.: 11/569,477

(22) PCT Filed: May 20, 2005
(86) PCT No.: PCT/CH2005/000286

§ 371 Date: Aug. 15, 2007

(30) Foreign Application Priority Data
May 24, 2004 (CH) PCTCH0400313

(51) Int. Cl. A61K 31/7016; C07H 1/00; C07H 1/06; A61P 7/06; C07H 3/04

(52) U.S. Cl. 514/53; 536/124; 536/127; 536/123.13

(57) ABSTRACT

Process for the preperation of iron-sucrose complex, in which
(a) an iron salt in aqueous solution is mixed with sucrose and an inorganic base, simultaneously or in any order, at a low temperature, in such a way that the reaction mixture has an acidity (pH) in the range 3<pH<12, and the reaction mixture is left at this acidity until all the iron salt has been converted to iron oxyhydroxide, wherein, when using an alkali metal hydroxide, the sucrose is always introduced at the beginning of the reaction or is added to the reaction mixture simultaneously with the alkali metal hydroxide;
(b) the acidity of the reaction mixture is then raised to a value in the range 10<pH<12 and the reaction mixture is heated until the desired iron-sucrose complex has completely formed, and the iron-sucrose complex formed is then precipitated by mixing with a suitable water-miscible solvent, the iron-sucrose complex being purified by removal of the anions present and any excess base, before or after the precipitation; the iron-sucrose complex prepared in this way; its use for the preparation of drugs; and drugs containing such an iron-sucrose complex.

The present invention relates to a process for the preparation of iron-sucrose complex, i.e. iron(II)-sucrose complex and iron(III)-sucrose complex, preferably iron(III)-sucrose complex. Iron(III)-sucrose complex can also be referred to as iron-sucrose complex, as sodium iron-sucrose or sodium iron(III)-sucrose, or as iron-sucrose or iron(III)-sucrose. The expression iron(III)-sucrose complex used in the description generally also includes iron(II)-sucrose complex.

Iron-sucrose complex, especially iron(III)-sucrose complex, and processes for its preparation are known per se. Iron(III)-sucrose complex is understood here as meaning the reaction product of iron oxyhydroxide, especially iron(III) oxyhydroxide, with sucrose, especially D(+)-sucrose. D(+)-sucrose is understood as meaning the sugar D-sucrose or the compound beta-D-fructofuranosyl-alpha-D-glucopyranoside. The expression “sucrose” or “D-sucrose” is also used. Iron(III)-sucrose complex contains organically bound iron and is used e.g. as a drug for raising the blood iron level in humans and animals.

The known processes have significant disadvantages. One of the main problems found in all the processes for the preparation of iron(III)-sucrose complex is the removal of the anion (e.g. the chloride content) resulting from the iron salt used (e.g. iron chloride), or the removal of the counterion formed, from the iron oxyhydroxide. This anion content is physiologically undesirable. In the known processes this chloride content is always removed immediately from the slurry of iron(III) oxyhydroxide. However, experience shows that it is very difficult to filter freshly precipitated colloidal iron oxyhydroxide. Although aged iron oxyhydroxide can easily be filtered off, it is unsuitable for synthesis of the physiologically active iron(III)-sucrose complex, so the iron oxyhydroxide obtained is slurried several times and the supernatant solution is decanted off. This procedure is technically impractical and expensive.

The present process according to the invention eliminates this disadvantage. In particular, it is not necessary to remove the chloride content (or other counterions or foreign salts) from the freshly precipitated colloidal iron oxyhydroxide. Further, the solid iron(III)-sucrose complex is obtained by simple precipitation, e.g. with the aid of an organic solvent, so the iron(III)-sucrose complex prepared according to the invention does not contain any unwanted carriers or additives. The iron(III)-sucrose complex is substantially easier and safer to handle as a solid than in the form of a solution, because the solid can be transported over long distances without decomposition and takes up a substantially smaller volume. In contrast to solutions, there is virtually no risk of microbiological contamination in the case of a solid. Likewise, a solid can be purified substantially better and unwanted by-products can be removed better than from a solution. It is therefore more advantageous to precipitate the product as a solid without the addition of any kind of foreign or auxiliary substances.

In the process according to the invention it is not necessary to add the base in portions at specific temperatures or to add different bases in a specific order. It suffices to heat the reaction mixture for formation of the iron(III)-sucrose complex, preferably at the reflux point. The solvent, i.e. the water, does not need to be evaporated off. To precipitate the product it suffices to add the solution to a water-miscible organic solvent, preferably an alcohol, or to add a water-miscible organic solvent, preferably an alcohol, to the solution, whereupon the iron(III)-sucrose complex formed in the solution precipitates out unchanged. The proportion of free sucrose in the precipitated product varies according to the concentration of the iron(III)-sucrose complex. When working in dilute solution, the solution is preferably concentrated until the residual concentrate has a good flowability. Concentration is not necessary when working in concentrated solution.

The process according to the invention is defined in the Claims. In particular, the present invention relates to a process for the preparation of iron-sucrose complex, especially iron(III)-sucrose complex, which is characterized in that

(a) an iron salt in aqueous solution, preferably an iron(III) salt and particularly preferably iron(III) chloride hexahydrate, is mixed simultaneously or in any desired order with sucrose, preferably D-sucrose, and an inorganic base, preferably an alkali metal carbonate and/or an ssssalkali metal hydrogen carbonate, preferably at a low temperature, i.e. at a temperature ranging from −10° C. to 40° C., preferably at about 5-25° C., so that the reaction mixture has an acidity (pH) in the range 3<pH<12, preferably in the range 5<pH<9 and particularly preferably of about 7, and the reaction mixture is left at this acidity until all the iron salt has been converted to iron oxyhydroxide, wherein, when using an alkali metal hydroxide, the sucrose is always introduced at the beginning of the reaction or is added to the reaction mixture simultaneously with the alkali metal hydroxide;

(b) the acidity (pH) of the reaction mixture is then increased to a value in the range 10<pH<12, preferably by adding an alkali metal hydroxide and/or an alkali metal carbonate and/or an alkali metal hydrogen carbonate and/or ammonium hydroxide and particularly preferably by adding an alkali metal hydroxide, especially sodium hydroxide, and the reaction mixture is heated until the desired iron-sucrose complex, preferably iron(III)-sucrose complex, has completely formed, preferably at a temperature ranging from 70° C. to the reflux point or, under pressure, at up to 140° C.; and the iron-sucrose complex formed is then precipitated by mixing with a suitable water-miscible solvent, preferably having a dielectric constant in the range 10-50 (at 20° C.), or with a mixture of such solvents, the iron-sucrose complex being purified by removal of the anions present and of any excess base, before or after the precipitation, in a manner known per se.

If, for example, the iron salt in aqueous solution is introduced simultaneously with the sucrose and then reacted with the inorganic base, it is assumed that iron oxyhydroxide forms as an intermediate, although the present invention is not bound to this explanation.

The procedure is preferably as follows:

(a1) An iron salt, preferably an iron(III) salt, in aqueous solution, preferably iron(III) chloride hexahydrate in aqueous solution, is mixed with an alkali metal carbonate, preferably 1.5-5.0 equivalents and particularly preferably 1.5-2.0 equivalents of the carbonate base, and/or with an alkali metal hydrogen carbonate, preferably 3.0-10.0 equivalents and particularly preferably 3.0-4.0 equivalents of a hydrogen carbonate base, in each case per equivalent of iron ion, preferably at a low temperature, so that the reaction mixture has an acidity in the range approx. 3<pH<12, preferably 5<pH<9, the reaction mixture is left at this acidity until all the iron salt, preferably iron(III) chloride hexahydrate, has been converted to iron oxyhydroxide, the iron oxyhydroxide formed is optionally prepurified by removal of the foreign ions (e.g. chloride ions), for example by means of decantation, ion exchange, filtration or ultrafiltration, and the requisite amount of sucrose, preferably at least 2 equivalents and particularly preferably 2.0-5.0 equivalents of sucrose, preferably D-sucrose, per equivalent of iron ion, is then added;

(b1) the acidity (pH) of the reaction mixture is then raised to a value in the range 10<pH<12, preferably by adding an alkali metal hydroxide and/or an alkali metal carbonate and/or an alkali metal hydrogen carbonate and/or ammonium hydroxide, preferably sodium hydroxide, and the reaction mixture is heated until the desired iron-sucrose complex, preferably iron(III)-sucrose complex, has completely formed, preferably at a temperature ranging from 70° C. to the reflux point, or, under pressure, at a temperature of up to 140° C., and the iron-sucrose complex formed is then precipitated by mixing with a suitable water-miscible organic solvent, preferably having a dielectric constant in the range 10-50 (at 20° C.), or with a mixture of such solvents, the iron-sucrose complex being purified by the removal of any chloride ions still present, before or after the precipitation, in a manner known per se.

Mixing with a suitable water-miscible organic solvent means that the solution is added to a water-miscible organic solvent, preferably having a dielectric constant in the range 10-50 (at 20° C.), or to a mixture of such solvents, or a water-miscible organic solvent, preferably having a dielectric constant in the range 10-50 (at 20° C.), or a mixture of such solvents, is added to the solution.

In terms of the present invention, the expression “a water-miscible organic solvent” means a water-miscible organic solvent or a mixture of different organic solvents which has a dielectric constant in the range 10-50 (at 20° C.), preferably in the range 20-50 (at 20° C.). This compound is preferably an alcohol or a ketone. If it is an alcohol, the compound is preferably a primary, secondary or tertiary C₍₁₋₆₎-alcohol, benzyl alcohol, ethylene glycol, propylene glycol or glycerol, preferably methanol, ethanol, n-propanol, isopropanol, n-butanol, sec-butanol and/or tert-butanol and particularly preferably methanol and ethanol. If it is a ketone, the compound preferably has the formula C₍₁₋₃₎-alkyl-C(O)—C₍₁₋₃₎-alkyl and particularly preferably the formula C_{(1,2)}-alkyl-C(O)—C_{(1,2)}-alkyl, and is preferably methyl ethyl ketone or acetone and particularly preferably acetone. “Mixture of such solvents” is preferably understood as meaning a mixture of the aforementioned solvents, preference being given to an aforementioned solvent or a mixture of such solvents which contains about 20% by weight, 40% by weight or 60% by weight of methanol or ethanol or about 20% by weight, 40% by weight or 60% by weight of a mixture of methanol and ethanol.

The present invention further relates to the iron-sucrose complex prepared in this way and to its use for the preparation of drugs, preferably for curing anaemic conditions. The present invention further relates to drugs which contain iron-sucrose complex prepared according to the invention.

According to the invention, the iron oxyhydroxide formed in the slightly acidic to moderately alkaline aqueous medium can be reacted directly with sucrose in situ, or after prior isolation, but without special purification by the removal of anions, e.g. chloride ions, to give iron-sucrose complex, preferably iron(III)-sucrose complex. In the particularly preferred embodiment, the iron oxyhydroxide formed in the slightly acidic to moderately alkaline aqueous medium is not isolated but reacted directly with sucrose in situ to give the iron-sucrose complex, the latter being obtained in colloidal solution. This solution can be prepurified, optionally after concentration, by the removal of unwanted constituents such as anions or excess base, by filtration, ultrafiltration, ion exchange, dialysis or another filtration technique known per se. Instead of iron(II) chloride hexahydrate or iron(III) chloride hexahydrate as starting material, it is also possible to use other appropriate iron salts, preferably iron(III) salts, although the use of iron(III) chloride hexahydrate has proved advantageous because this compound is cost-effective and easy to handle. Examples of such water-soluble iron salts are iron nitrates such as iron(III) nitrate hexahydrate or iron(III) nitrate nonahydrate.

The iron(III)-sucrose complex obtained after the precipitation can easily be further purified and/or washed to completely remove foreign substances (e.g. chloride). It is surprising, however, that the precipitation already affords a very pure product that satisfies the specifications, and a further purification is not normally necessary. A decisive feature is that, according to the invention, only the finished iron(III)-sucrose complex must be freed of any chloride ions present, which is appreciably easier than reprecipitating or slurrying the moist iron oxyhydroxide.

The precipitate of iron oxyhydroxide obtained in the process can be isolated or directly processed further. At least two equivalents of sucrose (per equivalent of iron ion) are added to the aqueous iron oxyhydroxide, and sodium hydroxide solution is added until the mixture gives a clearly basic reaction, preferably up to a pH of 10.0-12.0, and the mixture is heated at min. 70° C., preferably at the reflux point. It can optionally also be heated at up to 140° C. at elevated pressure. To obtain the desired properties of the product (molecular weight, pH, colour, acid stability), the mixture is heated at the reflux point for at least 0.2 hour, preferably 0.5-96 hours, particularly preferably about 1-8 hours and very particularly preferably about 6-8 hours. The desired complex, or the desired iron(III)-sucrose complex, forms in this process and is shown by analysis to be suitable for parenteral iron therapy. Optionally the iron(III)-sucrose complex formed is then purified by the removal of foreign salts by conventional methods, e.g. filtration, ultrafiltration, ion exchange, dialysis or another known method. The mixture is then optionally concentrated such that it still has a good flowability. After removal of the foreign salts, the aqueous solution of the resulting complex can be heated again, optionally before the precipitation and/or concentration, for a period of time required to achieve the objective, preferably at a temperature ranging from 70° C. to the reflux point or, under pressure, at a temperature of up to 140° C. This heating can be carried out e.g. in order to subject the product to steam sterilization, to concentrate the solution, to improve the flowability of the solution or to optimize the molecular weight of the product. However, by adding a water-miscible solvent or a mixture of such solvents, as defined above, the active substance can also be directly precipitated and filtered off or centrifuged off and optionally purified. The complex undergoes no further change during the concentration and precipitation.

The invention is illustrated by the following Examples of the preparation of the iron(III)-sucrose complex according to the invention.

### EXAMPLE 1

22.61 g (83.6 mmol) of iron chloride hexahydrate are dissolved in 80 ml of water at room temperature. A solution of 13.3 g (125.4 mmol) of sodium carbonate in 78.4 g of water is added, in a temperature range of −5° C. to 25° C., in such a way that the solution does not foam too vigorously. After about one third of the solution has been added, a flocculent precipitate begins to appear. After all the base has been added, the suspension formed is stirred until the evolution of gas ceases. 85.86 g (250.8 mmol) of D-sucrose and 27.03 g (202.7 mmol) of 30% aqueous sodium hydroxide solution are then added to the mixture, which is heated at the reflux point for three hours. The mixture is concentrated to 60% of its original volume and precipitated by addition to 860 g of methanol, with slow cooling at 20-40° C. The brown suspension formed is stirred overnight at room temperature and the product is filtered off and washed with methanol. It is dried at 50° C. under vacuum to give 45.52 g of a brown powder that is shown by analysis (gel permeation chromatography, inter alia) to be suitable for parenteral iron therapy. If the chloride content of the product is outside the specification, the product is subsequently slurried once in a 6/1 methanol/water mixture.

### EXAMPLE 2

22.61 g (83.6 mmol) of iron chloride hexahydrate are dissolved in 80 ml of water at room temperature. A solution of 13.3 g (125.4 mmol) of sodium carbonate in 78.4 ml of water is added, in a temperature range of −5° C. to 25° C., in such a way that the solution does not foam too vigorously. After about one third of the solution has been added, a flocculent precipitate begins to appear. After all the base has been added, the suspension formed is stirred until the evolution of gas ceases. 85.86 g (250.8 mmol) of D-sucrose and 27.40 g (205.5 mmol) of 30% aqueous sodium hydroxide solution are then added to the mixture, which is heated for two hours under pressure at 120-130° C. The mixture is concentrated to 60% of its original volume and precipitated by adding 573 g of methanol, with slow cooling at 20-40° C. The brown suspension formed is stirred overnight at room temperature and the product is filtered off and washed with methanol. It is dried at 50° C. under vacuum to give 45.85 g of a brown powder that is shown by analysis (gel permeation chromatography, inter alia) to be suitable for parenteral iron therapy. If the chloride content of the product is outside the specification, the product is subsequently slurried once in a 6/1 methanol/water mixture.

### EXAMPLE 3

22.61 g (83.6 mmol) of iron chloride hexahydrate are dissolved in 80 ml of water at room temperature. A solution of 13.3 g (125.4 mmol) of sodium carbonate in 78.4 ml of water is added, in a temperature range of −5° C. to 25° C., in such a way that the solution does not foam too vigorously. After about one third of the solution has been added, a flocculent precipitate begins to appear. After all the base has been added, the suspension formed is stirred until the evolution of gas ceases. 85.86 g (250.8 mmol) of D-sucrose and 19.87 g (149 mmol) of 30% aqueous sodium hydroxide solution are then added to the mixture, which is heated at the reflux point for 30 minutes. The mixture is filled into dialysis tubes and dialyzed overnight in a gentle stream of water. It is then concentrated to 35% of the original volume, refluxed for a further 6 hours and precipitated by adding 396 g of methanol, with slow cooling at 20-40° C. The brown suspension formed is stirred overnight at room temperature and the product is filtered off and washed with methanol. It is dried at 50° C. under vacuum to give 65.80 g of a brown powder that is shown by analysis (gel permeation chromatography, inter alia) to be suitable for parenteral iron therapy.

### EXAMPLE 4

19.79 g (73.2 mmol) of iron chloride hexahydrate are dissolved in 100 ml of water at room temperature. A solution of 11.43 g (107.8 mmol) of sodium carbonate in 100 ml of water is added, in a temperature range of −5° C. to 25° C., in such a way that the solution does not foam too vigorously. After about two thirds of the solution has been added, a flocculent precipitate begins to appear. After all the base has been added, the suspension formed is stirred until the evolution of gas ceases. 2.8 g (21 mmol) of 30% sodium hydroxide solution are then added and the precipitated solid is filtered off and washed with water (the solid does not need to be washed until free of chloride). The moist brown product, still containing a large amount of water, is slurried in 90 ml of water, and 60.12 g (175.6 mmol) of D-sucrose and 7.3 g (54.8 mmol) of 30% aqueous sodium hydroxide solution are added to the mixture, which is heated for 6 hours at the reflux point. The mixture is concentrated to 40% of its original volume and precipitated by addition to 500 g of methanol, with slow cooling at 20-40° C. The brown suspension formed is stirred overnight at room temperature and the product is filtered off and washed with methanol. It is dried at 50° C. under vacuum to give 48.5 g of a brown powder that is shown by analysis (gel permeation chromatography, inter alia) to be suitable for parenteral iron therapy.

### EXAMPLE 5

19.79 g (73.2 mmol) of iron chloride hexahydrate are dissolved in 100 ml of water at room temperature. A solution of 11.5 g (108.3 mmol) of sodium carbonate in 100 ml of water is added, in a temperature range of −5° C. to 25° C., in such a way that the solution does not foam too vigorously. After about two thirds of the solution has been added, a flocculent precipitate begins to appear. After all the base has been added, the suspension formed is stirred until the evolution of gas ceases. The precipitated solid is filtered off and washed with water (the solid does not need to be washed until free of chloride). The moist brown product, still containing a large amount of water, is slurried in 80 ml of water, and 60.48 g (176.8 mmol) of D-sucrose and 5.69 g (42.7 mmol) of 30% aqueous sodium hydroxide solution are added to the mixture, which is heated for 3 hours at the reflux point. The mixture is concentrated to 40% of its original volume and precipitated by addition to 113.3 g of ethanol, with slow cooling at 20-40° C. The brown suspension formed is stirred for 1.5 hours at room temperature and the product is filtered off and washed with ethanol. It is dried at 50° C. under vacuum to give 60.09 g of a brown powder that is shown by analysis (gel permeation chromatography, inter alia) to be suitable for parenteral iron therapy.

### EXAMPLE 6

178.11 g (659 mmol) of iron chloride hexahydrate are dissolved in 900 ml of water at room temperature. A solution of 166.2 g (1976 mmol) of sodium hydrogen carbonate in 2050 ml of water is added, in a temperature range of −5° C. to 25° C., in such a way that the solution does not foam too vigorously. Towards the end of the addition of the solution, a flocculent precipitate begins to appear. After all the base has been added, the suspension formed is stirred until the evolution of gas ceases. The precipitated solid is filtered off and washed with water (the solid does not need to be washed until free of chloride). The moist brown product, still containing a large amount of water, is slurried in 635 ml of water, 540 g (1579 mmol) of D-Sucrose are added and the pH is adjusted to 11.5-12.0 by the addition of 30% sodium hydroxide solution. The mixture is then heated for 8 hours at the reflux point. The mixture is concentrated to 40% of its original volume and precipitated by addition to 4350 g of methanol, with slow cooling at 20-40° C. The brown suspension formed is stirred for 2 hours at room temperature and the product is filtered off and washed with methanol. It is dried at 50° C. under vacuum to give 476 g of a brown powder that is shown by analysis (gel permeation chromatography, inter alia) to be suitable for parenteral iron therapy.

### EXAMPLE 7

The organic solvent used for the precipitation in Examples 1 to 6 above is n-propanol, isopropanol, n-butanol, sec-butanol, tert-butanol, benzyl alcohol, ethylene glycol, propylene glycol, glycerol, acetone or a mixture of these compounds, such as n-propanol, isopropanol, n-butanol, sec-butanol, tert-butanol, benzyl alcohol, ethylene glycol, propylene glycol, glycerol and acetone, containing 20% by weight, 40% by weight or 60% by weight of methanol and/or ethanol.

1. A process for the preparation of iron-sucrose complex, characterized in that
(a) an iron salt in aqueous solution is mixed with sucrose and an inorganic base, simultaneously or in any order, at a low temperature, in such a way that the reaction mixture has an acidity (pH) in the range 3<pH<12, and the reaction mixture is left at this acidity until all the iron salt has been converted to iron oxyhydroxide, wherein, when using an alkali metal hydroxide, the sucrose is always introduced at the beginning of the reaction or is added to the reaction mixture simultaneously with the alkali metal hydroxide;
(b) the acidity (pH) of the reaction mixture is then raised to a value in the range 10<pH<12, by the addition of an inorganic base selected from the group consisting of: alkali metal hydroxide, alkali metal carbonate, alkali metal hydrogen carbonate, ammonium hydoxide or combinations thereof, and the reaction mixture is heated until the desired iron-sucrose complex has completely formed, and the iron-sucrose complex formed is then precipitated by mixing with a suitable water-miscible solvent, the iron-sucrose complex being purified by removal of the anions present and any excess base, before or after the precipitation.

2. The process according to claim 1, characterized in that iron(III)-sucrose complex is prepared.

3. The process according to claim 1, characterized in that, in step (a), an iron(III) salt, preferably iron(III) chloride hexahydrate, is mixed with D-sucrose and an inorganic base, where said inorganic base is selected from the group of alkali metal carbonate, alkali metal hydrogen carbonate of combinations thereof at a temperature ranging from −10° C. to 40° C., so that the reaction mixture has an acidity in the range 5<pH<9, and the reaction mixture is left at this acidity until all the iron salt has been converted to iron oxyhydroxide.

4. The process according to claim 1, characterized in that, in step (b), the acidity of the reaction mixture is raised to a value in the range 10<pH<12 by the addition of an alkali metal hydroxide, and the reaction mixture is heated at a temperature in the range from 70° C. to the reflux point or, under pressure, at up to 140° C.

5. The process according to claim 1, characterized in that, in step (b), a water-miscible organic solvent or a mixture of such solvents having a dielectric constant in the range 20-50 (at 20° C.) is used for the precipitation of the iron-sucrose complex.

6. The process according to claim 5, characterized in that said water-miscible organic solvent is selected from the group of: an alcohol, a ketone or a combination thereof.

7. The process according to claim 5, characterized in that said water-miscible organic solvent is a ketone.

8. The process according to claim 6, characterized in that a mixture of these solvents containing about 20% by weight, 40% by weight or 60% by weight of methanol or ethanol or about 20% by weight, 40% by weight or 60% by weight of a mixture of methanol and ethanol, is used.

9. The process according to claim 1, characterized in that, in step (a), the iron salt is mixed with an alkali metal carbonate, 1.5-5.0 equivalents of the carbonate base, and/or with an alkali metal hydrogen carbonate, 3.0-10.0 equivalents of a hydrogen carbonate base, in each case per equivalent of iron ion, the reaction mixture is left at the resulting acidity until all the iron salt has been converted to iron oxyhydroxide, the iron oxyhydroxide formed is optionally prepurified by removal of the foreign ions, and at least 2 equivalents, of sucrose, per equivalent of iron ion, are then added.

10. The process according to claim 9, characterized in that the iron oxyhydroxide formed is prepurified by removal of the foreign ions by means of decantation, ion exchange, filtration or ultrafiltration.

11. The process according to claim 1, characterized in that the iron oxyhydroxide formed is reacted directly with sucrose in situ, or after prior isolation, but without special purification by the removal of anions, to give iron-sucrose complex.

12. The process according to claim 11, characterized in that the iron oxyhydroxide formed is reacted directly with sucrose in situ to give iron-sucrose complex.

13. The process according to claim 1, characterized in that, in step (b), the mixture is heated at the reflux point for at least 0.2 hour.

14. The process according to claim 1, characterized in that the colloidal solution containing the iron-sucrose complex is purified by the removal of foreign components by filtration, ultrafiltration, ion exchange, dialysis or another filtration technique known per se.

15. The process according to claim 1, characterized in that, after removal of the foreign salts, the aqueous solution of the resulting complex is heated at a temperature in the range from 70° C. to the reflux point or, under pressure, at a temperature of up to 140° C.

16. The process according to claim 1, characterized in that the complex obtained is directly precipitated, filtered off or centrifuged off and then purified.

17. Iron-sucrose complex prepared according to claim 1.

18. A process for the preparation of a drug, comprising the steps of:
providing an active ingredient for a drug; and
mixing said active ingredient with an iron-sucrose complex where said iron-sucrose is prepared by a process comprising the steps of:
providing an iron salt in aqueous solution:
mixing said iron salt in an aqueous solution, with sucrose and an inorganic base, simultaneously or in any order, at a low temperature, in such a way that the reaction mixture has an acidity (pH) in the range 3<pH<12, and the reaction mixture is left at this acidity until all the iron salt has been converted to iron oxyhydroxide, wherein, when using an alkali metal hydroxide, the sucrose is always introduced at the beginning of the reaction or is added to the reaction mixture simultaneously with the alkali metal hydroxide;
adjusting the acidity (pH) of the reaction mixture is then raised to a value in the range 10<pH<12, by the addition of an inorganic base selected from the group consisting of: alkali metal hydroxide, alkali metal carbonate, alkali metal hydrogen carbonate, ammonium hydoxide or combinations thereof;
heating the reaction mixture until the desired iron-sucrose complex has completely formed;
precipitating the iron-sucrose complex by mixing with a suitable water-miscible solvent; and
purifying the iron-sucrose complex by removal of the anions present and any excess base, before or after the precipitation.

19. Drugs containing an iron-sucrose complex prepared according to claim 1.

20. The drug prepared by the process of claim 18 for curing anaemic conditions.
